(19) United States
(12) Patent Application Publication
(10) Pub. No.: US 2025/0243463 A1
(43) Pub. Date: Jul. 31, 2025

(54) EXOSOMES DERIVED FROM HUMAN NASAL MUCOSA MESENCHYMAL STEM CELLS, METHOD FOR PREPARING EXOSOMES, AND APPLICATION OF EXOSOMES

(71) Applicant: JIANGNAN UNIVERSITY, WUXI (CN)

(72) Inventors: XIAOJIE LU, WUXI (CN); WENTAO SHI, WUXI (CN); LU BIAN, WUXI (CN)

(73) Assignee: JIANGNAN UNIVERSITY, WUXI (CN)

(21) Appl. No.: 19/037,082

(22) Filed: Jan. 25, 2025

(30) Foreign Application Priority Data
Jan. 30, 2024 (CN) 202410128148.7

(51) Int. Cl. C12N 5/0775; A61K 35/28

(52) U.S. Cl. CPC C12N 5/0668; A61K 35/28; C12N 2501/734

(57) ABSTRACT

The present invention provides exosomes derived from human nasal mucosa mesenchymal stem cells, their preparation method, and applications. The human nasal mucosa mesenchymal stem cells can be derived from the patients themselves, ensuring an adequate cell supply and strong plasticity. Research has found that as seed cells for extracting extracellular vesicles, these cells have been proven to promote the proliferation and differentiation of endogenous neural stem cells, bridge injury sites, and facilitate spinal cord injury repair. The effects are significant, making it convenient for widespread application and promotion.

### TECHNICAL FIELD

The present invention belongs to the field of biomedical technology, specifically relating to exosomes derived from human nasal mucosa mesenchymal stem cells and their preparation method and application.

### BACKGROUND

Spinal cord injury (SCI) is a common and significant clinical problem in orthopedics. How to effectively promote SCI repair is a major research focus and challenge in this field. The transplantation of extracellular vesicles (EVs) derived from ectomesenchymal stem cells (EMSCs) is one of the most promising therapeutic strategies for treating SCI.

Currently, the primary seed cells used for SCI repair transplantation include induced pluripotent stem cells (iPSCs), bone marrow mesenchymal stem cells (BMSCs), and adipose-derived stem cells (ADSCs). EVs derived from these cells have been proven to promote the proliferation and differentiation of endogenous neural stem cells (NSCs), bridge injury sites, and facilitate SCI repair. However, these cells have certain limitations in terms of acquisition.

iPSCs are generated by reprogramming terminally differentiated somatic cells through the introduction of specific transcription factors. This process requires a high level of technical expertise, making the large-scale acquisition of iPSCs challenging, and the purity of the obtained cells may be insufficient.

BMSCs, found in mammalian bone marrow stroma, possess multi-lineage differentiation potential, including differentiation into bone, cartilage, adipose, neural, and myogenic cells. BMSCs and their derivatives (such as exosomes) have been widely documented for their strong ability to promote SCI repair. However, acquiring BMSCs requires secondary surgical procedures, which pose a significant burden on donors. Additionally, BMSCs harvested from elderly donors exhibit lower activity and weaker proliferative capacity, making it difficult to obtain sufficient therapeutic exosomes.

ADSCs, obtained through liposuction, have high self-renewal and multi-directional differentiation potential, including differentiation into adipocytes, chondrocytes, myocytes, osteocytes, neurons, glial cells, and islet cells. ADSCs also secrete pro-angiogenic and anti-apoptotic factors, providing anti-inflammatory and antioxidant effects. However, the in vitro isolation and culture of ADSCs remain complex, making the acquisition of their derived EVs challenging.

Therefore, it is of great significance to explore new, more suitable stem cells (preferably autologous, with sufficient quantity and strong plasticity) as seed cells for EV extraction and autologous transplantation in SCI repair.

### SUMMARY

The present invention aims to address the challenges of seed cell acquisition, poor plasticity, and limited exosome yield associated with existing SCI repair techniques. It provides exosomes derived from human nasal mucosa mesenchymal stem cells (hNM-MSCs), their preparation method, and application. hNM-MSCs can be sourced from the patient's own nasal mucosa, ensuring an adequate supply of cells with strong plasticity. These cells serve as ideal seed cells for EV extraction and autologous transplantation for SCI repair, demonstrating significant efficacy and ease of application.

To achieve the above objective, the present invention provides the following technical solution:

A method for preparing exosomes derived from hNM-MSCs, comprising the following steps:

Step 1: Under sterile conditions, collect the middle turbinate nasal mucosa, finely chop the tissue, and perform enzymatic digestion using trypsin.

Step 2: Further fragment the digested mucosal tissue, perform centrifugation, and seed the fragmented tissue for culture for more than 40 hours.

Step 3: Collect the cell supernatant, perform another round of centrifugation, and obtain exosomes derived from hNM-MSCs.

The preparation method of the present invention is simple and facilitates large-scale production.

The invention also provides exosomes obtained by the above method.

The exosomes derived from hNM-MSCs are used in the preparation of products with neuroprotective effects against spinal cord injury.

These exosomes are also applicable for promoting spinal cord injury repair in vivo.

Additionally, they can be used to promote the differentiation of neural stem cells into neurons in vitro.

Furthermore, they aid in promoting endogenous neural stem cells' migration to the injury site and differentiation into neurons.

Another objective of the present invention is to provide pharmaceutical formulations containing these exosomes.

A pharmaceutical formulation comprising a therapeutically effective amount of hNM-MSC-derived exosomes and pharmaceutically acceptable excipients.

The formulation may include a medical carrier and diluents.

Furthermore, the pharmaceutical formulation may be an injectable preparation. A fibrin scaffold incorporating the exosomes derived from hNM-MSCs.

A functional product for SCI repair, including the pharmaceutical formulation or the fibrin scaffold.

Advantages of the present disclosure are as follows:

1. The provided preparation method is straightforward and scalable.

2. The derived exosomes are obtained from autologous nasal mucosa mesenchymal stem cells, ensuring ample supply and strong plasticity, making them effective for SCI repair and easy to apply clinically.

3. These exosomes have a broad range of applications, including neuroprotection, promoting neural differentiation, and facilitating endogenous neural stem cell migration and integration at the injury site.

4. The invention also provides pharmaceutical formulations and fibrin scaffolds incorporating these exosomes as functional products for SCI repair, enhancing their clinical applicability.

### BRIEF DESCRIPTION OF THE DRAWINGS

FIG. 1 is a detection diagram of in vitro isolation and culture of human-derived EMSCs in the embodiment.

FIG. 2 is an observation diagram of acute HE staining of the spinal cord in experimental animals.

FIG. 3 is a result diagram of EV transplantation.

FIG. 4 is a test result diagram showing that EVs promote the migration of endogenous neural stem cells to the injury site and their differentiation into neurons.

FIG. 5 is a test result diagram showing that EVs promote the differentiation of neural stem cells into neurons in vitro.

### DETAILED DESCRIPTION

Below, the present invention will be described in detail with reference to the accompanying drawings.

In order to make the objectives, technical solutions, and advantages of the present invention clearer, the following further elaborates on the invention with reference to the accompanying drawings and embodiments. It should be understood that the specific embodiments described herein are only intended to explain the invention and not to limit it.

Human nasal mucosa mesenchymal stem cells (EMSCs), exosomes (EVs)

### Embodiments

In vitro isolation and culture of human-derived EMSCs

Using a skull base endoscope, the nasal mucosa of the middle turbinate was collected under sterile conditions, approximately 100 mg, with a tissue size of 0.3×0.3 cm. The tissue was thoroughly minced, then digested with 0.25% trypsin at 37° C. for 10 minutes. After stopping the digestion, the tissue was further minced with ophthalmic scissors and centrifuged at 500 r/min. The minced tissue was then inoculated into a DMEM/F12 culture flask containing 10% wtFBS and cultured at 37° C., 5% CO2, and saturated humidity. The culture medium was replaced every 3 days. When the primary cells grew to approximately 75% confluence at the bottom of the flask, they were passaged for further culture.

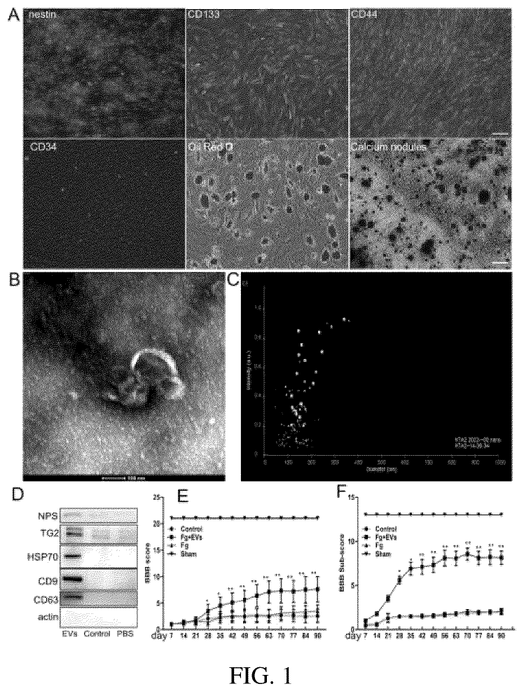

As shown in section A of FIG. 1, the in vitro isolated EMSCs highly express mesenchymal stem cell markers and neural crest markers, indicating that these cells are mesenchymal stem cells derived from the neural crest and possess multipotent differentiation capabilities. Section B of FIG. 1 shows EVs derived from EMSCs. Section C of FIG. 1 displays the size distribution of EV particles. Section D of FIG. 1 shows the characteristic proteins of EVs. Sections E and F of FIG. 1 respectively show the BBB and sub-BBB scores of EVs embedded in a fibrin scaffold in a rat spinal cord injury model within 90 days.

A rat acute SCI animal model was created using PinPoint™. Rats were anesthetized with 10% chloral hydrate, and a midline longitudinal incision was made at the level of the rat spine (T9-T12). The spinal periosteum was separated to expose the T10 vertebra, and the vertebral spinous process was removed with a high-speed drill to expose the spinal dura mater, ensuring the dura remained intact. The rat was fixed on a stereotaxic instrument. An impact hammer (3 mm in diameter) was placed in the spinal canal window, closely attached to the dura. Using the PinPoint™ brain injury impactor, a moderate controlled cortical impact injury model was established with the following impact conditions: impact speed of 2 m/s, impact depth of 2.5 mm, and contact time of 85 ms. After surgery, the wound was sutured. Exosomes derived from human nasal mucosa mesenchymal stem cells were intravenously pumped into the rats, with the same dose administered daily for one week.

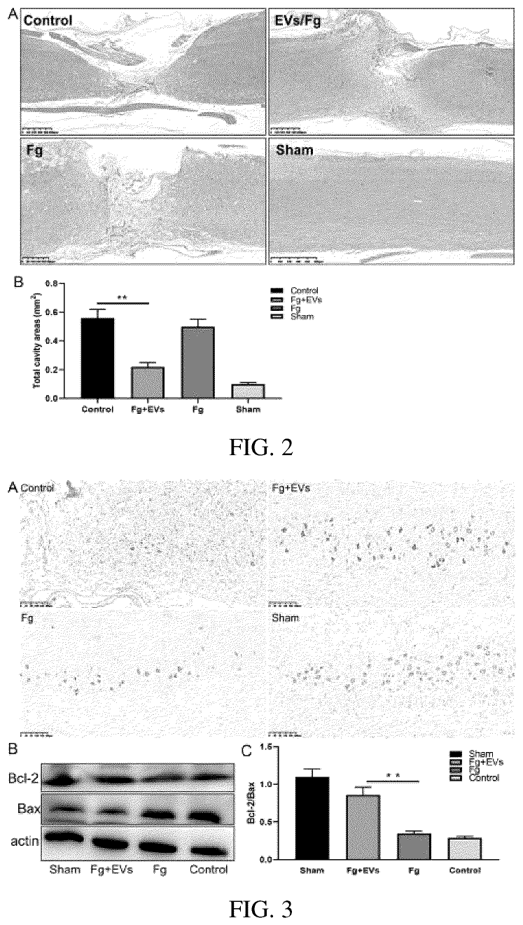

One week after treatment, the rats were euthanized, and tissue from the SCI site was collected. The tissue was fixed with paraformaldehyde, embedded in paraffin, sectioned, and stained with HE. Morphological changes in spinal cord nerve cells were observed under a microscope, as shown in FIGS. 2A and 2B, indicating that EVs promote spinal cord injury repair.

One week after treatment, the rats were euthanized, and tissue from the SCI site was collected for frozen sectioning and TUNEL staining. Apoptosis of spinal cord nerve cells was observed under a microscope, as shown in FIG. 3, demonstrating that EV transplantation can resist neuronal apoptosis after spinal cord injury.

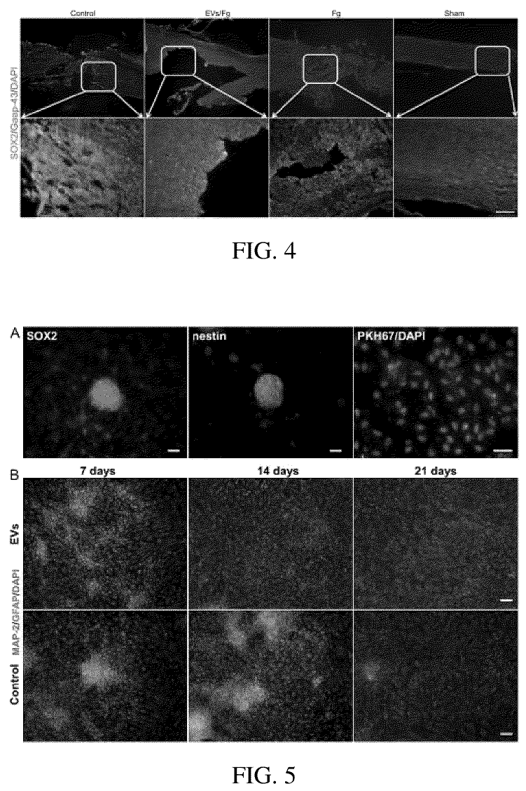

As shown in FIG. 4, EVs can promote the migration of endogenous neural stem cells to the injury site and their differentiation into neurons. FIG. 5 shows that EVs can promote the differentiation of neural stem cells into neurons in vitro.

Thus, the present invention provides exosomes derived from human nasal mucosa mesenchymal stem cells that can be used in the preparation of products with the following applications: resisting neuronal apoptosis after spinal cord injury, promoting the repair of spinal cord injury areas in vivo, promoting the differentiation of neural stem cells into neurons in vitro, and promoting the migration of endogenous neural stem cells to the injury site and their differentiation into neurons. These exosomes have broad applications, have been proven to promote the proliferation and differentiation of endogenous neural stem cells, bridge injury sites, and promote spinal cord injury repair. They are highly practical and easy to promote.

The above description is only a preferred embodiment of the present invention and is not intended to limit the invention. Any modifications, equivalent replacements, and improvements made within the spirit and principles of the present invention should be included within the scope of protection of the present invention.

1. A method for preparing exosomes derived from human nasal mucosa mesenchymal stem cells, characterized by comprising:
under sterile conditions, collecting nasal mucosa of middle turbinate, mincing it, and digesting it with trypsin;
mincing the digested mucosa again, centrifuging it, and then inoculating and culturing the minced tissue for more than 40 hours;
collecting the cell supernatant, centrifuging it again, and obtaining exosomes derived from human nasal mucosa mesenchymal stem cells.

2. Exosomes derived from human nasal mucosa mesenchymal stem, characterized in that the exosomes are obtained by the preparation method comprising:
under sterile conditions, removing the nasal mucosa of the middle turbinate, mincing it, and digesting it with trypsin;
mincing the digested mucosa again, centrifuging it, and then inoculating and culturing the minced tissue for more than 40 hours;
collecting the cell supernatant, centrifuging it again, and obtaining exosomes derived from human nasal mucosa mesenchymal stem cells.

3. Application of exosomes derived from human nasal mucosa mesenchymal stem cells in the preparation of a product for resisting neuronal apoptosis after spinal cord injury, wherein the exosomes are obtained by a method comprising:
under sterile conditions, removing the nasal mucosa of the middle turbinate, mincing it, and digesting it with trypsin;
mincing the digested mucosa again, centrifuging it, and then inoculating and culturing the minced tissue for more than 40 hours;
collecting the cell supernatant, centrifuging it again, and obtaining exosomes derived from human nasal mucosa mesenchymal stem cells.
